(19) United States
(12) Patent Application Publication
(10) Pub. No.: US 2007/0172490 A1
(43) Pub. Date: Jul. 26, 2007

(54) Leukocyte stimulation matrix

(75) Inventor: Martin Scholz, Oberursel (DE)

Correspondence Address: MH2 TECHNOLOGY LAW GROUP, 1951 KIDWELL DRIVE, SUITE 550, TYSONS CORNER, VA, 22182, US

(73) Assignee: LEUKOCARE GMBH, MUNICH (DE)

(21) Appl. No.: 10/591,470

(22) PCT Filed: Mar. 4, 2005
(86) PCT No.: PCT/EP05/02325

§ 371 Date: Sep. 1, 2006

(30) Foreign Application Priority Data
Mar. 4, 2004 (EP) 04005177.3

(51) Int. Cl. A61K 39/00; A61K 39/38; C12N 5/08

(52) U.S. Cl. 424/184.1; 435/372; 424/229.1

(57) ABSTRACT

The present invention relates to a leukocyte stimulation matrix for the simulation of leukocytes and/or the induction of an immunological tolerance comprising a) at least one carrier, b) a soluble matrix for embedding at least one component for generating a leukocyte stimulation and/or the induction of an immunological tolerance, and c) at least one component embedded into the soluble matrix for generating a leukocyte stimulation and/or the induction of an immunological tolerance; as well as to a module containing the matrix and the use thereof.

### FIELD OF THE INVENTION

The present invention relates to a leukocyte stimulation matrix, a leukocyte stimulation module comprising the leukocyte stimulation matrix, and to a process for stimulating leukocytes and/or the induction of immunological tolerance.

### BACKGROUND ART

The antigen-specific stimulation of leukocytes is an important and growing research field for the modulation of an immune reaction (vaccination, adoptive immune therapy etc.). Besides vaccination in the conventional sense there are presently known mainly therapeutic approaches for the ex vivo stimulation.

At present, there is no therapy sufficiently working for a number of chronical viral infectious diseases. The chronicity is based on the persistence of viral antigens in the tissue and the insufficient immune response thereto. Viral diseases are mostly treated with chemotherapeutica. This treatment often results in a resistance of the viruses and in strong side effects. Previous experiments regarding the ex vivo stimulation of dendritic cells and effector cells by dendritic cells (DC), e.g. in relation to tumour diseases, were only partly successful (Cerundolo et al., Nature Immunology, Bd. 5, Nr. 1, S. 7-10, 2004). The ex vivo stimulation of the effector cells and the subsequent return of the cells into the patient are presumably too susceptible to disturbance and too weak to induce clinically relevant effects. Moreover, in the methods presently employed the dendritic cells and the effector cells occurring only in a very small amount in the blood are isolated. The ex vivo expansion of these cells is a further step which is susceptible to disturbance.

The antigen-specific stimulation of leukocytes in whole blood (in vivo) has the advantage, in comparison to the ex vivo stimulation, that all physiological and essential factors of the blood necessary for the antigen-specific activation of leukocytes can be utilised.

WO 00/27999 discloses the embedding of hematopoietic precursor cells, antigen presenting cells and lymphoreticular stroma cells into a porous and solid matrix, wherein the matrix is covered with biological agents and is impregnated with a gel like agent. This matrix can be used for the induction of a T-cell reactivity in vitro.

WO 93/20185 discloses an in vitro process for the proliferation of precursors of dendritic cells.

WO 97/03186 (U.S. Pat. No. 6,121,044) discloses an implantable module having a matrix with embedded dendritic cells (DC) and being able to cause a primary and secondary immune response. This documents does relate to a module which can be used in vivo, however, the module has the disadvantage that the immune response by means of the module or the matrix disclosed therein has no possibility of time regulation. With this kind of stimulation it cannot be secured that, after the activation of the leukocytes has been carried out, the leukocytes can be released from the stimulated matrix in a controlled way to return them into the blood stream.

Banchereau and Steinmann describe in a review article in Nature, vol. 392, p. 245-252, 1998 dendritic cells and the participation in the regulation of the immune response.

WO 03/030965 discloses a module for the stimulation of leukocytes wherein a complex of an antigen presenting cell (MHC) and an antigen is immobilised on a carrier in said module. This document also discloses that a release of stimulated leukocytes takes place, however, it is not disclosed how such a release can be regulated in a controlled way.

WO 03/031473 discloses a module for reducing the activity of leukocytes, wherein the module comprises a carrier and a ligand which is bound to the carrier and which is capable of interacting with a receptor of leukocytes.

It is an object of the present invention to provide a matrix or a matrix-comprising apparatus that enables the stimulation of leukocytes in vivo and/or the induction of an immunological tolerance in the blood circulation, wherein the stimulated leukocytes can be released from the matrix in a time-controlled mode.

### SUBJECT MATTER OF THE INVENTION

The problem mentioned above is solved by the provision of a leukocyte stimulation matrix according to claim 1 and a leukocyte stimulation module according to claim 12.

The leukocyte stimulation matrix according to the present invention has the following components:
a) one or more carrier(s),
b) a soluble matrix for embedding one or more component(s) for generating a leukocyte stimulation and/or the induction of an immunological tolerance,
c) one or more component(s) embedded into the soluble matrix for generating a leukocyte stimulation and/or the induction of an immunological tolerance.

According to the present invention, circulating antigen-specific leukocytes bind to the components embedded into the soluble matrix, are bound for a while, are stimulated and are released as active cells from the matrix or the matrix-comprising module, respectively.

Such a leukocyte stimulation matrix according to the present invention can secure a successive and controlled dissolution of the outer matrix surface within a predetermined time range, preferably within a time range of from hours up to about one day. The soluble matrix is released layerwise in a controlled way into the leukocyte containing liquid, preferably whole blood, whereby also components contained in the soluble matrix can be released for generating a leukocyte stimulation and/or an induction of an immunological tolerance (e.g. antigens), and whereby leukocytes bound to these components are also released from the matrix. The introduction of a leukocyte stimulation matrix or a module containing the same, respectively, into the body results in a return of these leukocytes into the body via blood circulation. The dissolution time of the respective outer layers of the soluble matrix is selected such that a binding intensity and binding time suitable for the respective antigen-specific leukocyte activation is secured.

In a preferred embodiment of the present invention, one or more coupling components are provided in addition to components a) to c) in order to mediate the binding between the carrier and the one or more component(s) for generating a leukocyte stimulation and/or the induction of an immunological tolerance. This mediated binding is preferably a covalent binding so that at least part of the embedded components for generating a leukocyte stimulation and/or the induction of an immunological tolerance are covalently bound to the carrier via the coupling component. However, non-covalent bonds, e.g. ionic bonds, bonds on the basis of hydrophobic interactions, van der Waals interactions etc. of this component with the coupling component are also covered by the present invention. The present invention also embraces such embodiments wherein the component for generating a leukocyte stimulation and/or the induction of an immunological tolerance is bound to a coupling component and partly embedded into the soluble matrix without being bound to the coupling component.

Carrier

The carrier of the leukocyte stimulation matrix according to the present invention is not particularly limited as long as a soluble matrix according to the present invention can be applied on it. A carrier of a biocompatible material is preferred. Preferred are carriers having pores.

Materials, such as polyurethanes, polyamide or polyester can be used as duroplastic carrier materials, among which polyurethanes are preferred. Among the polyurethanes, hydrophilic materials with open pores as well as hydrophobic polyurethane foams can be used which optionally contain pigments such as carbon pigments and/or silicone pigments.

Furthermore, polyurethane varnishes or other semi-products can be used which optionally contain the above mentioned pigments. The medical grade (purchased from KCI) is particularly preferred. Materials, such as polycarbonates and polystyrene are suitable as thermoplastic carrier materials. Polyethylene or polypropylene are also usable, wherein these may preferably be used in combination with adhesive pigments. Elastomers are also usable.

Additional suitable polymers are e.g. PTFE (polytetrafluoroethylene), Dacron or polymethylpentane.

Further preferred materials are those which are used as dissolvable materials in surgery (sutures), e.g. Monocryl (poliglecaprone 25, PDS-2 (polydioxanon), Maxon (polyglyconate), Vicryl (polyglactin-910) and Dexon-Plus (polyglycolic acid). The use of such materials dissolvable in body fluids, such as whole blood, has the advantage that the carrier gradually dissolves after the complete dissolution of the outer matrix or coating, respectively, so that the leukocyte stimulation module or the leukocyte stimulation matrix used as a transplant do not necessarily have to be removed from the body.

Furthermore, glass in any possible forms, e.g. fibers, with open pores or foamed, is suitable as the carrier material.

Furthermore, metals are suitable as carriers, preferably by compatible metals or carriers covered with biocompatible metals. Natural materials such as gut skins or biological materials such as sponges can also be used preferably.

Polymer materials having pores are particularly preferred, in particular polyurethane. The pores can be of any size. Average pore sizes in the range of 0.5-2 mm, particularly about 1 mm, are preferred.

Soluble Matrix

A leukocyte stimulation matrix according to the present invention furthermore comprises a soluble matrix on the carrier, wherein one or more components embedded into the matrix for generating a leukocyte stimulation and/or the induction of an immunological tolerance are embedded. The term “soluble” as used herein means that the soluble matrix dissolves in whole blood within a time range of from hours to a few days.

In a preferred embodiment of the invention, the soluble matrix (b) is made of long chain sugar compounds such as starch, cellulose and/or glycogen, or it is made of polyethylene glycol. However, a long chain sugar compound does not have to be contained according to the present invention. Hence, PEG is a mandatory component of the soluble matrix in a preferred embodiment of the invention, and the long chain sugars are optional components. In a further preferred embodiment of the present invention, no long chain sugars are contained in the soluble matrix as components in addition to PEG.

In the above mentioned embodiment containing long chain sugar compounds, the soluble matrix preferably comprises 50-90 wt. %, more preferably 60-80 wt. % of one or more long chain sugar compounds and 10-50 wt. %, preferably 20-40 wt. % of a polyethylene glycol, based on the total of long chain sugar compounds and polyethylene glycol. In both of these embodiments of the soluble matrix according to the present invention, it can be secured that the soluble matrix slowly dissolves within about 4-12 hours in the blood. The dissolution can be slowed down in principle by an increase of the PEG content. Hence, a regulation of the dissolution time of the soluble matrix is possible through the variation of the contents of PEG and long chain sugar compounds.

The regulation of the dissolution time of the soluble matrix can furthermore be achieved through the variation of the molecular weight of PEG. Preferably, polyethylene glycol (PEG) is used in a molecular weight in the range of 1-200 kD. A molecular weight of from 10 to about 60 kD is preferred, a molecular weight of about 10 to 30 kD is more preferred, and about 30 kD is particularly preferred. Modified PEGs can also be used, e.g. those wherein PEG molecules are connected by means of a spacer. PEG is preferably used as an aqueous solution, wherein a solution of about 1-10 wt. %, preferably about 5 wt. %, of a PEG having e.g. a molecular weight of 15-20 kD is used. The concentration can be up to 20 wt. % of a PEG with a low molecular weight (e.g. about 6 kD), however, the concentration can also be lower for a PEG with a higher molecular weight. The suitable concentration can be determined by a skilled person by carrying out tests. PEG can also be bound to various cytokines, e.g. interferon (IFN), wherein such PEG interferon products can also be used as a component of the soluble matrix according to the present invention. Thus, it is possible to incorporate cytokines as a leukocyte stimulating agent into the soluble matrix.

### Component for Generating a Leukocyte Stimulation and/or the Induction of an Immunological Tolerance

According to the present invention, leukocyte stimulation means that previously conditioned immune cells are specifically enhanced in their immune response. The term leukocytes comprises B-lymphocytes, T-lymphocytes, granulocytes and neutrophils.

The term induction of a tolerance as used herein means that an anergy of the leukocytes is induced toward a specific antigen, which means that an inactivation takes place. Leukocytes are stimulated with an antigen and simultaneously, co-stimulating molecules are inhibited.

According to the present invention, molecules such as antigens, haptens, MHC molecules, co-stimulating factors, cell components and/or membrane fragments of antigen presenting cells can be used as components for generating or triggering or producing respectively, a leukocyte stimulation and/or induction of an immunological tolerance. The antigens do not have to be isolated previously. Unaffected or mostly unaffected viruses, bacteria, cells or coatings thereof containing antigens can also be used as a component for generating a leukocyte stimulation and/or the induction of an immunologic tolerance. Inactivated viruses, bacteria etc. can also be used. The inactivation can be carried out by known processes, e.g. UV-irradiation. The antigens, such as peptides, that are isolated e.g. from preparations of viruses, bacteria or tumour cells, can be coupled to MHC molecules or to the membrane components of antigen presenting cells, e.g. dendritic cells (DC), of the patient to be treated (autologous cells), or they can be derived from allogenic donors. The immunologically relevant content of the soluble matrix, i.e. the part that causes an immune response, can be referred to as immune stimulating complex (IK) according to the present invention.

The antigen can be a synthetic antigen, or the antigen can be collected from viruses, bacteria, fungi, protozoae, parasites (e.g. worms), tumours, allergens, cell cultures or from a body's own tissue. The MHC molecule and/or the co-stimulating factor can be collected from a body's own tissue, from cell cultures, or they can be produced synthetically.

An overview of co-stimulating molecules can be found e.g. in Rothstein and Sayegh, Immunological Reviews 2003, “T-cell co-stimulatory pathways in allo-graft reaction and tolerance”. These molecules are incorporated into the present invention by reference.

The following co-stimulating molecules can be used in the presence of an antigen according to the present invention:
All co-stimulating molecules of the CD28/CTLA-4:B7-family: CD28, CTLA-4, ICOS, PD-1, B7-1, B7-2, B7RP-1, PD-L1, PD-L2.
Tumour necrosis factors: Tumour necrosis factor receptor family:
   CD154/CD40L, 4-IBB (CD137), Ox-40 (CD134), CD27; Ligands: CD40, 4IBBL (CD137L), Ox40L (CD134L), CD70.

Further co-stimulating molecules which will be found in the future are also covered.

Those components for generating a leukocyte stimulation and/or the induction of an immunological tolerance e.g. against a virus, a virus of the family of Herpes viruses, in particular Cytomegalo virus (CMV), Epstein Barr virus (EBV) or Herpes Simplex virus (HSV 1+2), or a fragment or part thereof, a virus coating or a virus coating fragment thereof, containing antigens which cause an immune response are preferred.

Furthermore, the following components can be used: SARS (Corona virus); Rhino virus; Picorna viruses, in particular Polio virus, Coxsackie virus, Retro viruses, in particular the human immune deficiency virus (HIV), a hepatitis causing virus, in particular hepatitis B virus (HBV) or hepatitis C virus (HCV), Corona viruses, in particular SARS associates Corona viruses; and/or the respective antigens thereof or a fragment or part thereof, a virus coating or a virus coating fragment thereof containing antigens that cause an immune response.

Furthermore, all other viruses associated with chronic inflammatory diseases or tumour diseases, orthomyxo viruses (influenza), paramyxo viruses (mumps, measles in case of no or insufficient vaccination), papova viruses (papilloma viruses) can be used according to the present invention. Viruses against which there is no vaccination and/or with acute pathogenicity/lethality can also be used.

The leukocyte stimulation matrix is in particular suitable fcr patients who do not respond to an antiviral chemotherapy (because they e.g. have developed resistance) or in the case where a systemic treatment of local inflammations caused by viruses is not successful (e.g. CMV retinitis).

Particularly preferred is the antigen of a Cytomegalo virus of the strain CMV Hi91, AD169, Towne, Davis or a coating, a part or a fragment thereof. However, other laboratory strains or wild type strains are also covered by the present invention.

RNA viruses, DNA viruses, viruses with walls, viruses without coating, onkogenic viruses (papilloma virus), HHV-8 etc. can be used as virus types.

In the case of an incompatibility toward antibiotics, resistances or local inflammations induced by bacteria, bacteria or parts or fragments thereof which contain antigens can be used according to the present invention.

Basically all species from the families or genuses, respectively, of staphylococci; streptococci; enterococci; neisseriae; enterobacteriae; vibrionae (Cholera); all non-fermenting bacteria; campylobacter; helicobacter; haemophilus; bordetellae; legionellae; all microorganisms causing anthropozoonose; corynebacteriae; bacillus; clostridiae; mykobacteria; nocardiae; treponemae; borreliae (preferred is an immediate test on patients with borreliose); leptospirae; bartonella; mykoplasmae; chlamydiae are usable.

Among the fungi, plastomyces (candida); hyphomycetes (molds, Aspergillus); dimorphous fungi; and others, such as Pneumocystis carinii are particularly usable, in particular humanpathogenic fungi and so-called “hospital microorganisms”, such as Aspergillus or Candida albicans.

Among the parasites, protozoae, trematodae, cestodae or nematodae are particularly usable according to the present invention.

Prions or other unknown or not defined pathogens are basically usable.

In the case of tumours, membrane components of inactivated tumour cells, in particular of malign melanomas, can be used preferably.

In the case of auto immune diseases/allergies, basically all relevant antigens presently known or known in the future, in particular collagen, cell membranes of biliary epithel cells etc. can be used.

A preferred inhibitory factor that can be used for the generation of immune tolerance is LIR-1.

The concentration of the component for generating a leukocyte stimulation and/or the induction of an immunologic tolerance in the coating can be determined by the skilled person using suitable tests. Preferably, the amount or concentration of this component upon incorporation into the body corresponds to the amounts used in conventional vaccination. In conventional vaccination, e.g. about 20 μg of virus antigen are used.

The overall composition of the soluble matrix together with the embedded antigen in a preferred embodiment of the present invention is approximately as follows:

Definition of the matrix (wt. %):

| | possible range | preferred range |
|---|---|---|
| total of PEG and | 99.5-99.999 wt. % | 99.9-99.99 wt. % |
| long chain sugar | | |
| antigen | 0.001-0.5 wt. % | 0.01-0.1 wt. % |

In case a whole virus, bacterium, a coating thereof or the like is used in place of an essentially isolated antigen, the above mentioned antigen content can also be more than 0.5 wt. % or 0.1% wt. %.

As mentioned above, a long chain sugar is not necessarily present. The proportion of PEG and long chain sugar compound in a preferred embodiment is 50-90 wt. %, preferably 60-80 wt. % of long chain sugar compound(s) and 10-50 wt. %, preferably 20-40 wt. % of polyethylene glycols, based on the total of long chain sugar compounds and polyethylene glycol.

Coupling Component

In a preferred embodiment of the present invention, a coupling component is used to mediate the binding between the carrier and the one or more component(s) for generating a leukocyte stimulation and/or the induction of an immunological tolerance.

This coupling component preferably mediates a covalent bond and is preferably an element selected from cyanogen bromide, cyanoboro hydride, agarose, ararose derivatives, silane, silane derivatives or a combination thereof. In a less preferred embodiment, p-toluene sulfonyl chloride can be used. Particularly preferred is an alkoxysilane, even more preferred is a anhydro-alkoxysilane or another alkoxysilane having at least one carboxyl group. The alkoxy group preferably is a methoxy or ethoxy group. A particularly preferred anhydroalkoxysilane is 3-(triethoxy silyl)propyl succinic acid anhydride (GENIOSIL® GF 20, Wacker). Amino group containing alkoxysilanes are also usable, e.g. (3-aminopropyl)trimethoxysilane or [3-(2-aminoethylamino)propyl]trimethoxysilane. Further preferred is (3-(2,3-epoxy propoxy) propyl)trimethoxysilane (GENIOSIL® GF 80). Further alkoxysilanes can be selected in accordance with the respective carrier and the conditions.

It has been found that alkoxysilane, in particular anhydride alkoxysilane such as GENIOSIL® GF 20, but also GENIOSIL® GF 80, are particularly suitable for mediating the binding to a carrier of polyurethane or glass.

The invention allows a time limited binding of leukocytes from whole blood or of leukocytes isolated from whole blood, their specific antigen stimulation and the recycling of the stimulated leukocytes into the blood circulation by dissolution of the matrix in the whole blood or a leukocyte containing physiological liquid, respectively.

Furthermore, the induction of an immunological tolerance is possible according to the present invention by the addition of inhibitory factors and concurrent specific activation. The addition of inhibitory factors can e.g. be carried out during the formation of a soluble matrix, this means the inhibitory factors are embedded into the soluble matrix as it is formed, or the addition can be carried out during the leukocyte stimulation. The inhibitory factors can also be embedded into the soluble matrix through one of the above mentioned coupling components. In the case of an induction of an immunological tolerance a leukocyte is brought into contact with the antigen, however, it cannot be active due to the influence of the inhibitory factors against the antigen. A suitable inhibitory factor is e.g. LIR-1. Further inhibitory factors are known to the skilled person and are covered by the present invention. Inhibitory factors that are yet to be found are also covered by the present invention.

The leukocyte stimulation matrix according to the present invention can also be used as an implant.

Leukocyte Stimulation Module

The leukocyte stimulation matrix according to the present invention can be used in a leukocyte stimulation module. The leukocyte stimulation module comprises a housing preferably made of glass or plastic, wherein the plastic is preferably non-toxic and chemically inert towards a biological liquid such as whole blood, which means that it is essentially insoluble. The leukocyte stimulation module comprises at least one opening. Preferred is at least one inlet opening and at least one outlet opening, more preferably exactly one inlet opening and exactly one outlet opening. The housing or module, respectively, can also be provided in a further embodiment, e.g. having only one opening through which the leukocyte containing liquid flows in and also flows out. Further embodiments of the module can be designed by the skilled person, e.g. embodiments having more than one opening, such as two inlet openings and/or outlet openings.

Furthermore, the present invention relates to a process for the stimulation of leukocytes and/or the induction of an immunological tolerance, wherein a leukocyte containing liquid, such as whole blood, is contacted with a leukocyte stimulation matrix according to the present invention. This contacting is preferably carried out in a leukocyte stimulation module according to the present invention. The contacting is carried out more preferably in the blood circulation of the patient, i.e. in vivo.

The module according to the present invention can be used in patients or in clinical situations, respectively, deficient in the cellular immune response e.g. towards viral or bacterial infections or tumour antigens.

The module according to the present invention can be introduced e.g. into the blood circulation of the patient. In a preferred embodiment, the module is introduced into the blood circulation of a patient through a Sheldon catheter for a transient introduction of the module into the patient. In that case, circulating antigen-specific T-cells bind to the components embedded into the matrix, are bound there for a while, are stimulated and leave the module as highly active cells. Effector cells with only insufficient specific function are stimulated and leave the module as highly active cells. Memory cells previously brought into contact with a component embedded into the soluble matrix also bind to the matrix. These conditioned cells are also highly activated. Furthermore, non-conditioned (naive) T-cells can bind to the matrix and are conditioned in respect of the presented antigen MHC-molecule, co-stimulating factor or other cell component. The co-stimulating factors which can optionally be added to the module or which can be embedded into the soluble matrix (e.g. DC-adhesion molecules, cytokines etc.) cause an activation of these cells which are recycled into the blood circulation in order to eliminate the pathogenic agent via specific effector mechanisms in the blood circulation. The induction of a humoral immune response may follow (T-cell mediated B-cell activation).

An immune tolerance can be achieved as described above by using a respective matrix in which inhibitory factors are embedded, or by adding inhibitory factors into the module during leukocyte stimulation.

Bound and stimulated leukocytes are released due to the dissolution of the soluble matrix and enter the body. Simultaneously, deeper layers of the matrix containing antigenic determinants appear on the surface during the dissolution of the matrix, and new and non-occupied binding sites become free. The antigen mediated leukocyte binding and stimulation proceeds continuously until there is no soluble matrix anymore.

It is an advantage that the use of the module according to the present invention allows the accumulation of leukocytes and an immunostimulatory mechanism in a defined volume. In an ex vivo use according to the conventional art, stimulated leukocytes are recycled into the body of the patient, while the distribution of the stimulated leukocytes is totally unclear after their return into the patient.

The use of the leukocyte stimulation module (i.e. in particular by introducing it into the blood stream of the patient) or the ex vivo incubation of the matrix with the whole blood of the patient results in the binding of circulating leukocytes, e.g. T-cells, on the outer layer of the soluble matrix. The composition of the soluble matrix according to the present invention results in the successive dissolution of the outer layers. Hence, bound and stimulated leukocytes are also released from the solid and are recycled into the blood circulation by the effect of the blood stream or by mechanical treatment (e.g. shaking). The dissolution of the outer layer of the wall results in a renewal of the immunological surface. Leukocytes can bind to the immuno-stimulatory complexes, antigens, cell components, MHC molecules etc. newly appearing on the surface. This process is repeated until the wall is completely dissolved. The dissolved components are non-toxic and not hazardous in the blood since they exclusively are biocompatible materials and/or the patient's autologous components. The possibly continuing immunogenicity of the circulating immunostimulatory complexes in the blood is an additional advantage, since there is a further antigen-specific immunostimulation in the patient until these components are degraded biologically.

The module preferably has a plastic housing with a volume of preferably about 5 to 500 ml. The inlet and outlet nozzles are adjusted in their diameter to the tube connections of the catheter. The catheter can have two lumina (e.g. a Sheldon catheter), so that it is possible to reduce the flow speed of the blood per lumen by up to 50%. The housing contains the leukocyte stimulation matrix according to the present invention.

If necessary, after filling the leukocyte stimulation module filling with the patient's blood it can be disconnected from the catheter connection as a functional unit for carrying out e.g. further modifications ex vivo (e.g. the addition of co-stimulatory factors, cytokines, hormones, inhibitory factors etc.) without having to remove the blood from the module. The housing filled with blood can be incubated above 30° C. for several hours under continuous moving and can subsequently be returned to the patient through the catheter directly from the leukocyte stimulation module. This operation can be repeated several times.

### ADVANTAGES OF THE INVENTION

There is presently no method for the time controlled antigen-specific leukocyte stimulation in the blood flow or in the whole blood, respectively, or generally in leukocyte-containing liquids. The present invention solves the problem that the stimulation of leukocytes by immobilised antigens according to the prior art results in such a stable binding to the solid body rendering a release of the leukocytes impossible. This may result in an accumulation of leukocytes within the module used according to the prior art. This results in an increase of the resistance and in a specific stress for the leukocytes, in undesired side reactions and increased clogging effects. The continuous recycling of the stimulated leukocytes into the blood circulation according to the present invention enables the solution of the above mentioned problems.

The leukocyte stimulation module or the leukocyte stimulation matrix, respectively, according to the present invention can be used in the therapy against chronical viral infectious diseases. The chronicity is based on the persistence of the antigen in the tissue and the insufficient immune response thereto. The viral diseases are mostly treated with chemotherapeutica. This therapy often results in the formation of reminiscence of the kidneys and to severe side effects. The present invention can secure an induction or enhancement, respectively, of a highly specific immune response against a pathogenic agent de novo.

The binding to the component(s) for generating a leukocyte stimulation and/or induction of an immunological tolerance can result in an enhancement of the immune response of previously conditioned immune cells. These cells present in low concentration in the blood do not have to be isolated with much effort and do not have to be expanded ex vivo, since the cells automatically enter the leukocyte stimulation module via the blood stream and are stimulated in this module. After their activation, the leukocytes directly enter the blood circulation and the tissue as effector cells by dissolution of the soluble matrix. The physiological environment in the blood flow secures that all factors necessary for maturing the highly active effector cells are present.

Moreover, the present invention allows a new immunisation and the induction of tolerance.

The present invention furthermore relates to the use of a leukocyte stimulation matrix or a leukocyte stimulation module according to the present invention for leukocyte stimulation and/or the induction of an immunological tolerance as well as the use in methods for the detection of the distribution of active T-cell subtypes or for vaccination.

The present invention will be described in the following by means of examples which, however, are not intended to restrict the scope of the invention.

1. Embodiment Relating to Antigens Embedded in the Soluble Matrix

The Cytomegalo virus strain Hi91 was cultivated in human cultivated foreskin fibroblasts. After the occurrence of the cyto-pathological effects, the cell culture supernatants were collected, the viruses were concentrated by ultra-centrifugation, washed with a phosphate buffered salt solution (0.1 M PBS with Ca²⁺, Mg²⁺, pH 7.4) and inactivated by means of UV irradiation. The viruses were incubated for a short time (3 to 5 minutes) at room temperature (PEGilated) with 100 μl sodium bicarbonate buffer (50 mM; pH 8.0-9.6) containing 5 wt. % of 15-20 kD PEG (product no. P2263, Sigma-Aldrich, 2,2′-([methylethylidene]-bis[4,1-phenylene-oxymethylene])-bis-oxiran-polymer with α-hydroxypoly(oxy-1,2-ethandiyl). 10 mg of polyurethane foam with open pores (PU Medical Grade, KCI) were incubated together with the PEGilated viruses contained in the reaction mixture at room temperature and were then dried.

Subsequently, 20 μl of the antigen preparation (CMV Hi91) were admixed thereto. The antigen of 1×10⁶-1×10⁷ viruses were used for the stimulation tests. Then, whole blood (500 μl) of a CMV positive donor with known detectable and persistant CMV specific cellular immune activity was then added to the pre-treated samples, and an incubation was carried out for one hour under moving at 37° C.

After the carrier was washed with the above mentioned sodium bicarbonate buffer, the blood was prepared for the cytometric through flow analysis (FACS) for the detection of de novo generated pro-inflammatory cytokines in T-cells (CD4 and CD8). A commercially available standard test kit (Becton Dickinson) was used for the detection of the T-cell activation (CD69/IFN-gamma).

2. Embodiments Having Covalently Bound Antigens

The test was carried out as described above in section 1. However, the antigen was not added together with PEG, but the PU foam was pre-treated with PEG and the cellulose for 1 to 2 hours with the coupling component 3-(triethoxysilyl)propyl succinic acid anhydride ((5% Geniosil® GF20 in Methanol) prior to contacting. Subsequently, antigens from the coating of the Cytomegalo virus pre-treated as described above were covalently bound to the carrier material by incubation.

Results:

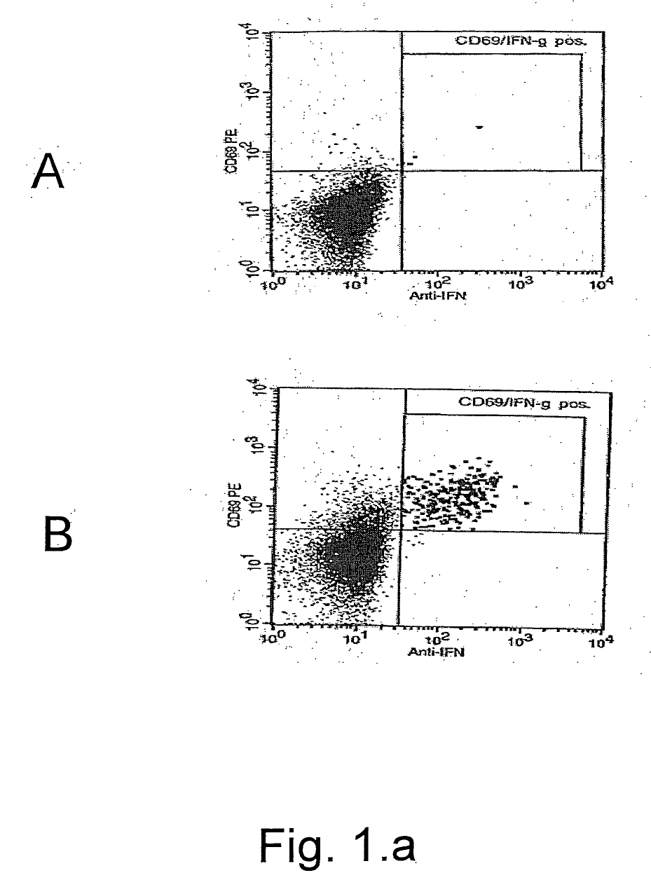

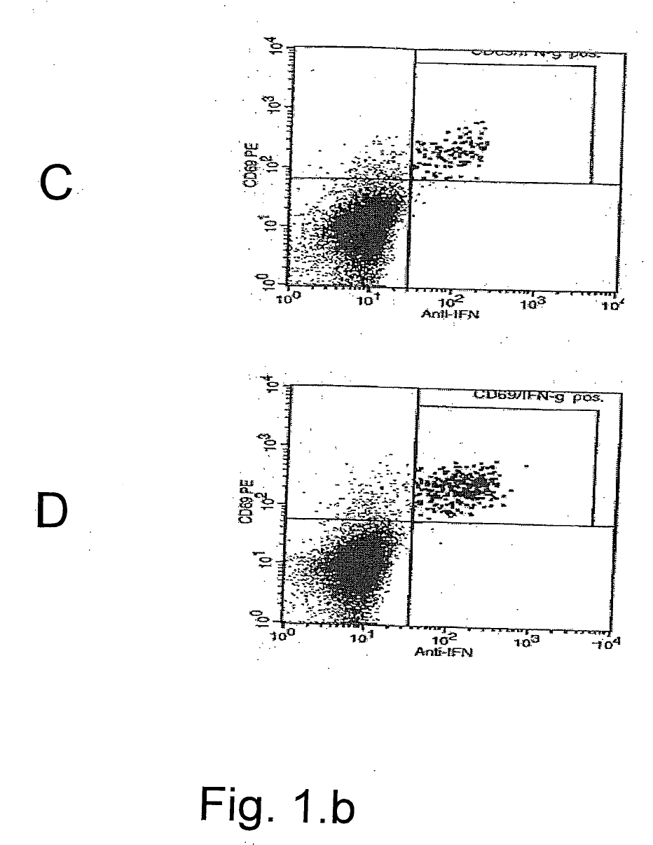

Both covalently bound antigens and antigens embedded in the soluble matrix could induce a T-cell mediated immune response in whole blood. The carrier with the covalently bound antigens showed a decreasing immune reaction (CD69/IFN-γ) in assays successively repeated several times. In contrast thereto, the generated immune reaction induced by a soluble matrix was constant over time. FIG. 1 shows an example of the test results of the cytometric through flow tests for the stimulation of the CMV-specific immune response (activation marker CD69/Interferon-gamma production) by covalently bound CMV-antigens (polyurethane KCI Medical Grade with silane (5% Geniosil® in methanol) pre-treated) as well as by non-covalently embedded CMV-antigens. The release of cytokines was inhibited by the pre-treatment of the cells with Brefeldin A. The quadrantic analysis shows a specific activation of 2.49% of the lymphocytes (CD4⁺) after subtraction of the control values (0.02% of unstimulated cells).

Hence, antigens covalently bound to the carrier or embedded in a soluble matrix can induce a specific T-cell mediated immune response in whole blood. The soluble matrix results in a continuous renewal of the antigen stimulating component which is in contact with the blood. Blood components can therefore not inhibit the functional capacity.

FIG. 1.a. and b.: The cytometric through flow tests, shown as quadrantic analysis. The dots in the upper right part each represent the cells activated by CMV-HI91-antigen (CD69/IFN-γ)CD4+ in the whole blood of a healthy CMV positive donor.
A) unstimulated control: 0.02%
B) CMV-Hi91-antigen on polyurethane treated with silane: 2.49%
C) CMV-Hi91-antigen on polyurethane treated with silane (repetition): 0.75%
D) CMV-Hi91-antigen on soluble matrix (after repetition): 2.02%

The above tests were repeated with other carriers, e.g. sepharose, glass and polystyrene, which showed similar results.

1. Leukocyte stimulation matrix for the stimulation of leukocytes and/or the induction of an immunological tolerance having the following components:
a) at least one carrier,
b) a soluble matrix for embedding at least one component for generating a leukocyte stimulation and/or the induction of an immunological tolerance,
c) at least one component embedded into the soluble matrix for generating a leukocyte stimulation and/or the induction of an immunological tolerance.

2. Leukocyte stimulation matrix according to claim 1, further comprising at least one component for mediating the binding between the carrier and the at least one component for generating a leukocyte stimulation and/or the induction of an immunological tolerance.

3. Leukocyte stimulation matrix according to claim 2, wherein the binding is a covalent binding.

4. Leukocyte stimulation matrix according to claim 1, wherein the at least one component for generating a leukocyte stimulation and/or the induction of an immunological tolerance is selected from the group consisting of antigens, MHC molecules, co-stimulatory factors, cell components, cell coatings, bacteria, viruses and combinations thereof.

5. Leukocyte stimulation matrix according to claim 4, wherein the at least one component for generating a leukocyte stimulation and/or the induction of an immunological tolerance is a synthetic antigen or is obtained from viruses, bacteria, fungi, tumours, allergens, endogenous tissue, and/or the MHC molecule; and
the co-stimulatory factors are obtained from endogenous tissue, cell cultures and/or synthetically.

6. Leukocyte stimulation matrix according to claim 4 wherein the at least one component for generating a leukocyte stimulation and/or the induction of an immunological tolerance is a virus of the family of herpes viruses or a fragment thereof.

7. Leukocyte stimulation matrix according to claim 1, wherein the carrier is selected from the group consisting of polyurethanes, polycarbonates, polystyrene, dissolvable materials used in surgery, glass, natural materials, biological materials, and combinations thereof.

8. Leukocyte stimulation matrix according to claim 2, wherein the at least one coupling component is selected from the group consisting of cyanogen bromide, cyanoboro hydride, agarose, agarose derivatives, silane, silane derivatives, and combinations thereof.

9. Leukocyte stimulation matrix according to claim 8, wherein the silane derivative is an alkoxy silane.

10. Leukocyte stimulation matrix according to claim 1, wherein the soluble matrix is made of long chain sugar compounds selected from the group consisting of starch, cellulose, and glycogen or polyethylene glycol.

11. Leukocyte stimulation matrix according to claim 10, wherein the soluble matrix is made of 50-90 wt. % of a long chain sugar compound and 10-50 wt. % of polyethylene glycol, based on the total of long chain sugar compound and polyethylene glycol.

12. Leukocyte stimulation module comprising a housing with at least one opening and a leukocyte stimulation matrix according to claim 1.

13. Leukocyte stimulation module according to claim 12 comprising at least one inlet opening and at least one outlet opening.

14. A process for the stimulation of leukocytes and/or the induction of an immunological tolerance wherein a leukocyte containing liquid is contacted with a leukocyte stimulation matrix according to claim 1.

15. A process according to claim 14, wherein the contacting is carried out in a leukocyte stimulation module according to claim 12.

16. (canceled)

17. (canceled)

18. Leukocyte stimulation matrix according to claim 6, wherein the at least one component for generating a leukocyte stimulation and/or the induction of an immunological tolerance is a cytomegalo virus or a fragment thereof.

19. Leukocyte stimulation matrix according to claim 7, wherein the natural material is a gut skin.

20. Leukocyte stimulation matrix according to claim 7, wherein the biological material is a sponge.

21. Leukocyte stimulation matrix according to claim 9, wherein the alkoxy silane is at least one of an anydroxyalkyoxy silane and another alkoxy silane comprising at least one carboxyl group.

22. Leukocyte stimulation matrix according to claim 11, wherein the soluble matrix is made of 60-80 wt. % of a long chain sugar compound and 20-40 wt. % of a polyethylene glycol, based on the total of long chain sugar compound and polyethylene glycol.

23. Leukodyte stimulation module according to claim 13, comprising one inlet opening and one outlet opening.

24. A method for the stimulation of leukocytes and/or the induction of an immunological tolerance comprising providing a leukocyte stimulation matrix according to claim 1.

25. A method for detecting distribution of activated T-cell subtypes or for vaccinations comprising providing a leukocyte stimulation matrix according to claim 1.
